(19) United States
(12) Patent Application Publication
(10) Pub. No.: US 2018/0256363 A1
(43) Pub. Date: Sep. 13, 2018

(54) CAGE FOR DISC SPACE BETWEEN VERTEBRAE

(71) Applicant: Soojung MOON, Lewisville, TX (US)

(72) Inventor: Soojung MOON, Lewisville, TX (US)

(21) Appl. No.: 15/879,010

(22) Filed: Jan. 24, 2018

Related U.S. Application Data
(60) Provisional application No. 62/451,046, filed on Jan. 26, 2017.

(51) Int. Cl. A61F 2/44; A61F 2/30

(52) U.S. Cl. CPC A61F 2/4465; A61F 2/30771; A61F 2002/30777; A61F 2002/30133; A61F 2002/30827; A61F 2002/30617; A61F 2002/3085; A61F 2002/30785; A61F 2002/30158; A61F 2002/30156; A61F 2002/30878

(57) ABSTRACT

The present invention relates to a cage for a disc space between vertebrae, and more particularly, to a cage for a disc space between vertebrae, which is capable of performing a rotating motion to be stably inserted when being inserted between the vertebrae.

Disclosed is a cage for a disc space between vertebrae, including: a cage body inserted between vertebrae; and a pivoting member rotatably connected to the cage body and fastened with an internal rod of a cage inserting device, in which the pivoting member includes a connection portion connected with the cage body and a threaded portion which extends from an end portion of the connection portion and protrudes to the outside of the case body and screw-fastened with the internal rod.

### CROSS REFERENCE TO RELATED APPLICATION

This application claims the benefit of U.S. provisional patent application No. 62/451,046 filed Jan. 26, 2017, the entire disclosure of which is incorporated herein by this reference.

### FIELD OF THE INVENTION

The present invention relates to a cage for a disc space between vertebrae, and more particularly, to a cage for a disc space between vertebrae, which is capable of performing a rotating motion to be stably inserted when being inserted between the vertebrae.

### BACKGROUND OF THE INVENTION

An interverbal disc as a disc of cartilage sandwiched between vertebrae of a spine performs an absorption function of absorbing a load and impact of a body between respective spines except for a part of a cervical vertebra and distributing the impact like a spring. In this case, the interverbal disc serves to hold the spine not to be separated, smooth a range of spinal articulation by separating two spines so as to prevent spinal nerves from being compressed, and make a motion of each spine.

Such an interverbal disc consists of a fibrous ring and a nucleus pulposus. The fibrous ring regulates the motion of spinal fragments and an inner nucleus consists of 70 to 80% of water. The interverbal disc cushions or transmits the load and the impact applied in a vertical direction. In degenerative disc diseases, the fibrous ring becomes weaker in their motions or an ability to accept a nucleus and a water content of the fibrous ring decreases. A complex result, leads to diseases such as spinal stenosis, osteophyte formation, disc prolapse, and nerve root compression.

As one method of treating the disease accompanied by the interspine disc, there may be a method of replacing a space between two adjacent spine supports with an artificial disc or implant, a so-called cage, after removing an interspine disc of a human body, which is damaged. That is, the method is to restore the function of the spine by restoring an original distance between two adjacent spinal supports, which is an original height of the interspine disc in order to create a natural state as possible by implanting the cage.

In recent years, transforaminal lumbar interbody fusion (TLIF) has been proposed as a surgical procedure for inserting such a cage into the spines. The TLIF as one of spine body fusion techniques is a surgical method of inserting the cage in a posterior approach. When the TLIF is specifically described, the TLIF as an operation of inserting the cage by using an insertion device while removing a spinal joint in a direction in which a neuropore comes out after the spine is incised to be small along both sides of spinal muscles and the spine is minimally exposed to fix screws has an advantage in that bleeding is fewer and an operation time can be shortened.

In the TLIF, for the minimum incision and minimization of interference in the human body, in general, a tip of the cage is first operated so as to be inserted through a rear surface (a back side) of the human body and positioned between the spines and thereafter, a lateral surface of the cage is disposed on a front surface (an abdomen side of the human body) between the spines to complete insertion of the cage. That is, an impactor, which is an auxiliary device, is required to make the lateral surface of the inserted cage face the front surface between the spines and by applying a force to the lateral surface of the cage using the impactor, and as a result, the cage can be arranged by rotating the cage. However, the operation using the impactor has a disadvantage in that it is difficult to make the operation or the success or the failure of the operation depends on an operation ability of the operator.

As a method for facilitating the adjustment of the position of the cage, there have been proposed technologies such as Korean Patent Registration No. 10-1273199 (hereinafter, referred to as ‘Patent Document 1’) and Korean Patent Registration No. 10-0371308 (hereinafter, referred to as Patent Document 2) have been proposed. The cage inserting devices of the prior art are configured to implement articulated rotational motion, the so-called articulating mechanism in which the tip of the cage is inserted facing the front surface between the spines and then, the cage is pivoted.

In the cage inserting device in which the articulating mechanism is implemented, it is very important to maintain a standing posture of the cage without rotating the cage during the insertion of the cage. In addition, it is very important that the cage can be stably inserted by enhancing coupling force between the insertion devices while minimizing a height (thickness) of the cage according to the requirements and in order to solve these technical problems, various researches and developments are performed.

### REFERENCE

### Patents

1. Korean Patent 10-1194219 (Oct. 18, 2012)
2. Korean Patent 10-0371308 (Jan. 23, 2003)

### DESCRIPTION OF THE INVENTION

### Problems to Solve

An object of the present invention is to provide a cage for a disc space between vertebrae which implements an articulated rotary motion and enhances coupling force with a cage inserting device to be stably inserted.

The technical objects of the present invention are not limited to the aforementioned technical objects, and other technical objects, which are not mentioned above, will be apparently appreciated by a person having ordinary skill in the art from the following description.

### Solution for the Problems

In accordance with an embodiment of the present invention, disclosed is a cage for a disc space between vertebrae, including: a support unit inserted between vertebrae; a connection portion connected with the support unit, and a pivoting member including an insertion device connecting unit which extends from an end portion of the connection portion to protrude to the outside of the support unit and connected with an insertion device.

In accordance with the cage for a disc space between vertebrae, the connection portion may be hinge-connected to the support unit by a pin member.

In accordance with the cage for a disc space between vertebrae, a slot providing a pivoting space of the pivoting member may be formed at a rear end of the support unit, and a first structure reinforcing unit supported by a first moving unit of the cage inserting device and a second structure reinforcing unit supported by a second moving unit of the cage inserting device may be formed in the slot.

In accordance with the cage for a disc space between vertebrae, the connection portion may be connected to a cage body by the pin member, and the pivoting member and the pin member may be made of a material having a higher strength than the cage body. For example, the cage body may have a PEEK material and the pivoting member and the pin member may have a titanium alloy material.

In accordance with the cage for a disc space between vertebrae, the insertion device connecting unit may include a male thread fastened with a female thread formed in the cage inserting device.

In accordance with the cage for a disc space between vertebrae, first and second markers having a pin shape may be installed in the cage body and the pint member is disposed in a triangular shape together with the first and second markers to serve as a positioning marker.

In accordance with the cage for a disc space between vertebrae, the first and second structure reinforcing units may be formed to have predetermined angels, respectively with respect to a coupling direction of the cage inserting device and for example, the first structure reinforcing unit may be formed to be inclined at an angle of 45 degrees with respect of the coupling direction of the cage inserting device and the second structure reinforcing unit may be formed to be inclined at an angle of 90 degrees with respect of the coupling direction of the cage inserting device.

In accordance with the cage for a disc space between vertebrae, a concave-convex pattern is repeatedly formed on the surface of the cage body and the concave-convex pattern may be formed in a triangular shape when viewed from the front-rear direction along the insertion direction of the cage housing and formed in a trapezoidal shape when viewed from a lateral direction vertical to the front-rear direction.

### Advantage of the Invention

According to an exemplary embodiment of the present invention, since a height of a case body is minimized and a diameter of a threaded portion of a pivoting member can be increased through a structure in which the pivoting member rotatably connected to the cage body protrudes to the outside of the cage body, stronger coupling force with a cage inserting device is provided.

Further, according to the exemplary embodiment of the present invention, first and second structural reinforcing units are formed in slots of the cage body at predetermined angles, thereby implementing the articulated rotary motion and enabling stable insertion in initial insertion.

In addition, according to the exemplary embodiment of the present invention, it is possible to facilitate the insertion of the cage at the time of the initial insertion through a concavo-convex pattern structure of a shape considering an insertion direction of the cage and prevent forward and backward deviation between vertebrae after seating the cage.

### BRIEF DESCRIPTION OF THE DRAWINGS

FIG. 1 is a perspective view of a cage for a disc space between vertebrae according to an exemplary embodiment of the present invention;

FIG. 2 is an exploded perspective view of the cage for a disc space between vertebrae illustrated in FIG. 1;

FIG. 3 is a plan view of the cage for a disc space between vertebrae illustrated in FIG. 1;

FIG. 4 is a cross-sectional view of the cage for a disc space between vertebrae illustrated in FIG. 3;

FIG. 5 is a perspective view of a device for inserting a cage for a disc space between vertebrae according to an exemplary embodiment of the present invention;

FIG. 6 is a cross-sectional view illustrating an operational state of the cage inserting device of FIG. 5;

FIG. 7 is a diagram illustrating a positional change of a first structure reinforcing unit depending on rotation of the cage;

FIG. 8 is a diagram illustrating insertion mechanism of the cage during the operation of cage inserting deice;

FIG. 9 is a front view of the cage for a disc space between vertebrae, which shows a shape of a concave-convex pattern illustrated in FIG. 1; and

FIG. 10 is a side view of the cage for a disc space between vertebrae, which shows a shape of a concave-convex pattern illustrated in FIG. 1.

### DETAILED DESCRIPTION OF THE INVENTION

Hereinafter, a cage for a disc space between vertebrae related with the present invention will be described in more detail with reference to drawings.

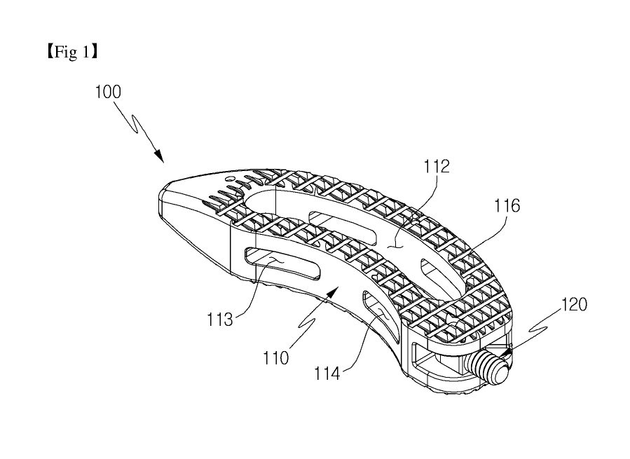

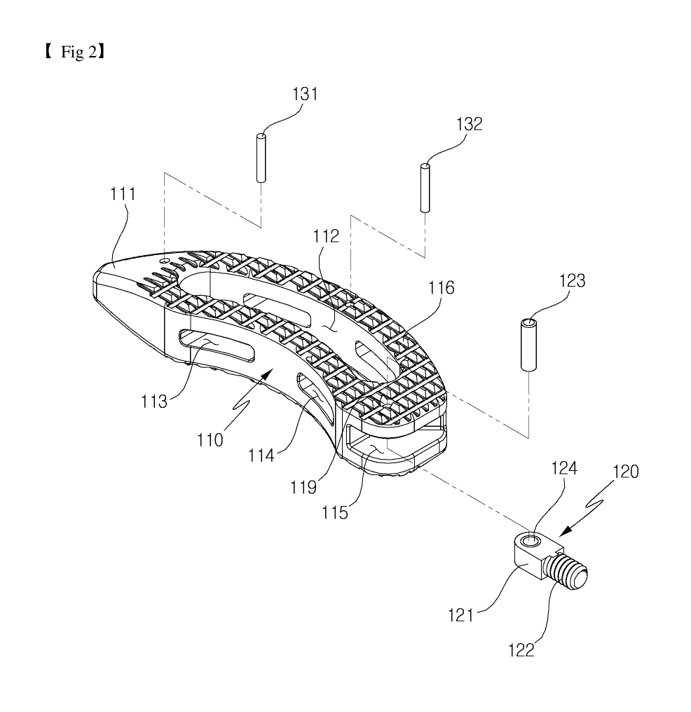

Referring to FIGS. 1 to 3, the cage 100 for a card disc between vertebrae according to an exemplary embodiment includes a support unit 110 and a pivoting member 120 rotatably connected to a slit formed on one surface of the body.

The support unit 110 forms an external appearance of the cage 100 and, in particular, has a constant thickness, i.e., a thickness close to an undamaged disc, to be inserted between the vertebrae. The support unit 110 has a shape suitable for anatomical insertion between the vertebrae. That is, a side portion positioned on an abdomen side of a human body after insertion is convex and a side portion located on a back side of the human body may have a rounded shape on the whole in a concave shape.

An introduction portion 111 of the support unit 110 as a portion into the cage is first inserted when being inserted into the human body may be formed to be inclined and may be formed in a vertically symmetrical shape so that insertion is easy. A concavo-convex pattern 116 may be repeatedly formed on upper and lower surfaces of the support unit 110 to prevent slippage when being in contact with a vertebral body 10. The concave-convex pattern 116 serves to prevent the slippage and further strengthen fusion with the vertebral body 10. The shape of the concave-convex pattern 116 will be described below in detail.

In the support unit 10, a hollow bone receiving hole 112 receiving bone fragments penetrating may penetrate at the center of the support unit 110 and a plurality of through-holes 113 and 114 may be formed on a periphery surrounding the hollow portion 112 so as to more smoothly receive the bone fragments. The bone fragments may be smoothly received even in a cage housing through the side through-holes 113 and 114 and creation of a void of (empty space) may be effectively prevented by increasing a density of the received bone fragments of the cage.

The pivoting member 120 is rotatably connected to a rear end of the support unit 110. A slot 115 is provided at a rear end opposite to the introduction portion of the support unit 110 and the pivoting member 120 is connected to a cage body 11 so as to be rotatable within the slot 15.

The pivoting member 120 includes a connection portion 121 connected to the support unit 110 and a threaded portion 122 extending from an end portion of the connection portion 121. The connection portion 121 may be formed in such a manner that the end portion thereof is rounded to be suitable for a pivotal motion and is hinge-connected to the support unit 110 by a pin member 123. An insertion hole 124 into which the pin member 123 is inserted may penetrated in the connection portion 121 and the pin member 123 is penetrated and inserted into the insertion hole 124 of the connection portion 120 through a fastening hole 119 formed in the support unit 110 to form a hinge connection structure of the connection portion 121.

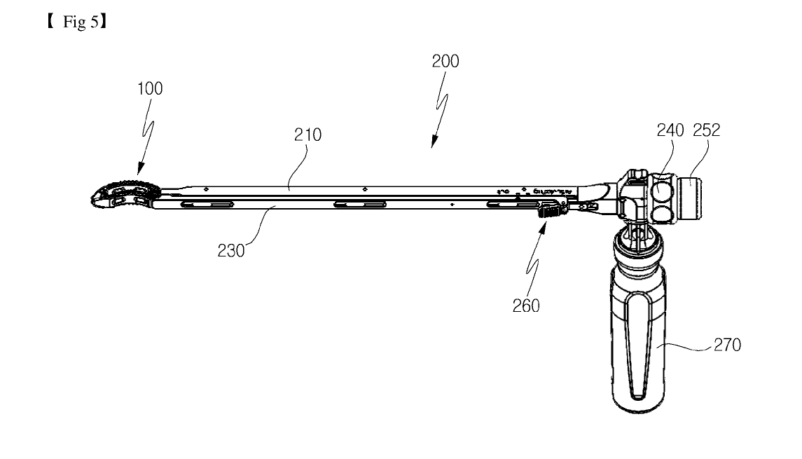

The threaded portion 122 is formed to extend from the end portion of the connecting portion 121 and protrude to the outside of the case body 110 and is configured to be fastened with an internal rod 250 of a cage inserting device 200 (see FIG. 5) to be described below.

The threaded portion 122 has a shape in which a thread is formed in a cylindrical member extending from the connection portion 121. The threaded portion 122 includes male threads that are fastened to female threads formed in the internal rod 250. However, the present invention is not limited thereto, and it is also possible to form the female threads in the threaded portion 122 and to form the male threads on the hollow inner rod 250 inside a housing of the cage inserting device.

Through a structure in which the threaded portion 122 of the pivoting member 120 protrudes to the outside of the support unit 110, the height of the support unit 110 may be minimized and the diameter of the threaded portion 122 may be increased (for example, a thread of M3.0 or more is usable) without being limited to the thickness of the case body 110, and as a result, coupling force with the cage inserting device 200 separately provided, in detail, the internal rod 250 may be further enhanced, thereby enabling stable insertion when the cage 100 is initially inserted. Since the pivoting member 120 is connected to the cage and remains inserted between the vertebrae even after a procedure is completed, applying the male screws to the threaded portion 122 will be more appropriate in terms of reducing the diameter.

Meanwhile, according to the embodiment, the pivoting member 120 and the pin member 123 which hinge-connects the pivoting member 120 to the housing may be made of a material having a higher strength than the support 110. For example, the support unit 110 may be made of a resin material having biocompatibility with the human body and a property value close to a bone such as polyetheretherketone (PEEK) and the pivoting member 120 and the pin member 123 may be made of a titanium alloy (e.g., Ti6Al4V ELI) material. With such a configuration, the pivoting member 120 and the pin member 123 may perform a function of supporting a vertical load higher than the strength of the peak material. Further, by using the titanium alloy material, it is possible to provide an additional fusion effect with the bone fusion material.

Meanwhile, a first marker 131 and the second marker 132 are installed on the support unit 110 and the first and second markers 131 and 132 enable easily determining a position and a posture of the cage 100 during operation through radiation. The first and second markers 131 and 132 may have a radiation-impermeable material, for example, a tantalum material.

The pin member 123 for hinge-connecting the pivoting member 120 is also formed of the radiation-impermeable material (for example, a titanium alloy) to serve as a marker. In the case of the embodiment, the first marker 131 is positioned at the introduction portion of the support unit 110, the second marker 132 is positioned at one side of the center of the support unit 110, and the pin member 123 is positioned at the rear end of the support unit 110 to form a triangular structure connecting the first maker 131, the second marker 132, and the pin member 123.

According to such a structure, the position and the posture of the cage 10 may be determined according to the shape of the triangle formed by the first marker 131, the second marker 132, and the pin member 123 detected by the radiation. In the case of the embodiment, the pin member 123 serves as both a connection means and the marker, which is advantageous in that the number of markers required for manufacturing the cage may be reduced.

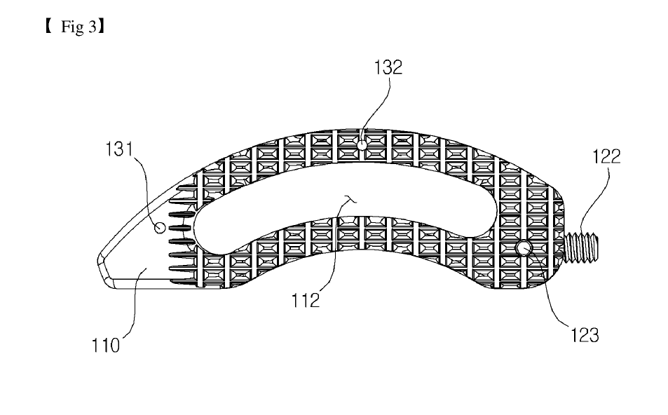

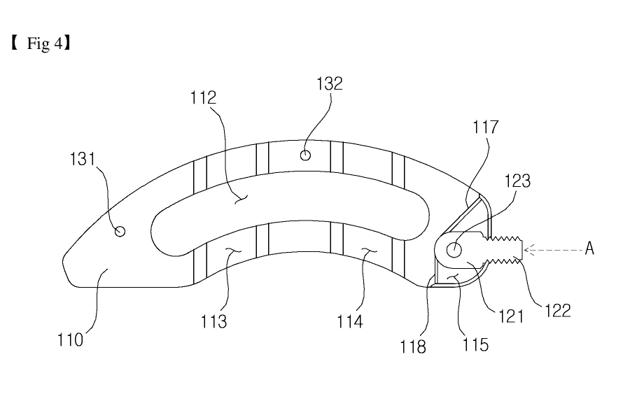

FIG. 4 is a cross-sectional view of the cage for a disc space between vertebrae illustrated in FIG. 3.

Referring to FIG. 4, a first structure reinforcing unit 117 and a second structure reinforcing unit 118 may be formed in the slot 115 formed at the rear end of the support unit 110. The first structure reinforcing unit 117 is supported by a first moving unit 220 of the cage inserting device 200 to be described later and the second structure reinforcing unit 118 is supported by a support bar 230.

The first structure reinforcing unit 117 and the second structure reinforcing unit 118 are formed so as to have predetermined angles, respectively with respect to a coupling direction of the cage inserting device 200, that is, an axial direction which follows the initial insertion direction of the cage.

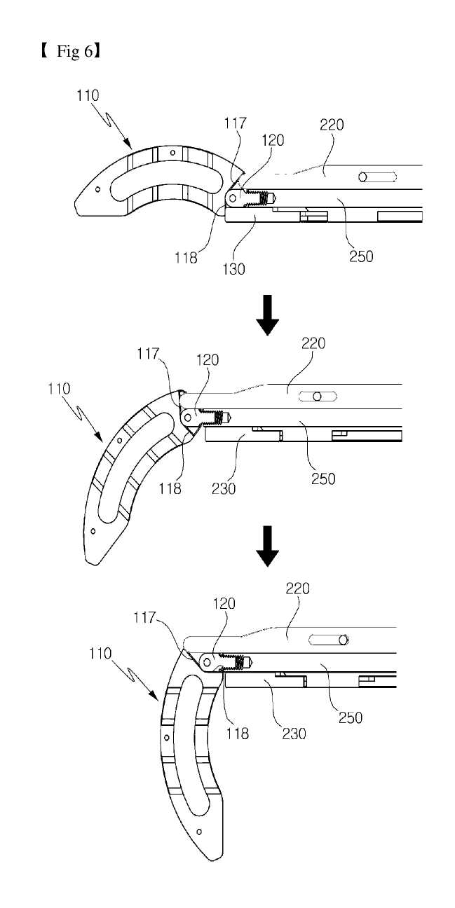

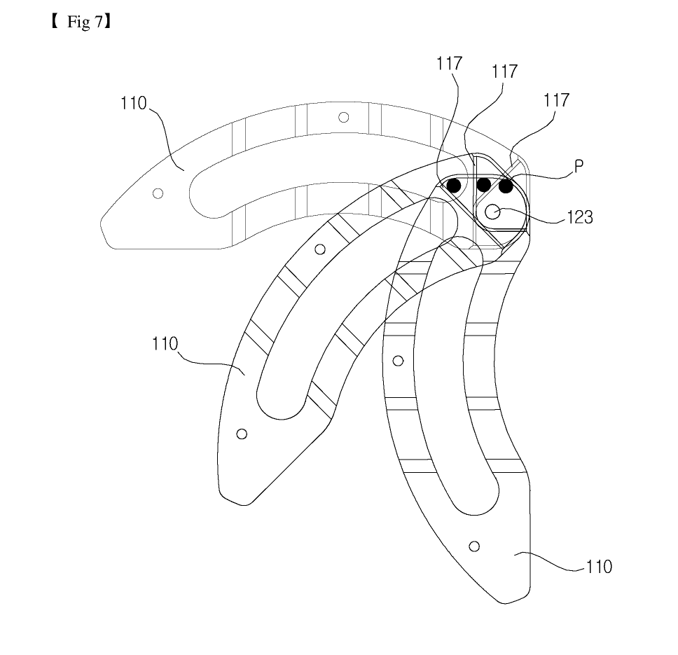

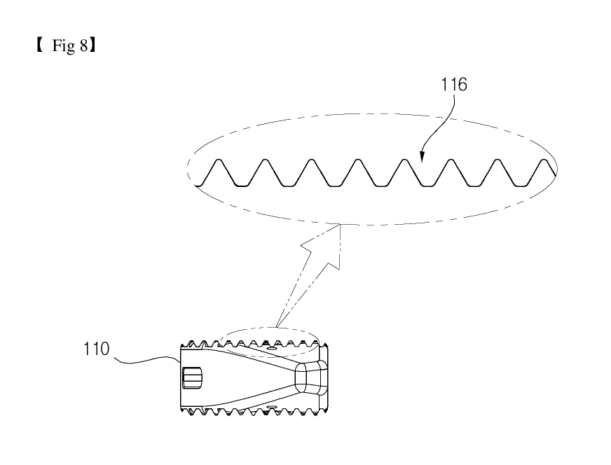

FIG. 5 is a perspective view of a device for inserting a cage for a disc space between vertebrae according to an exemplary embodiment of the present invention and FIG. 6 is a cross-sectional view illustrating an operational state of the cage inserting device of FIG. 5. In addition, FIG. 7 is a diagram illustrating a positional change of a first structure reinforcing unit depending on rotation of the cage., and FIG. 8 is diagram illustrating insertion mechanism of the cage during the operation of cage inserting device.

Referring to FIGS. 5 to 6, the cage for a disc space between vertebrae according to an embodiment includes a support unit 210, a first moving unit 220, a second moving unit 230, and an adjustment unit 240.

The support unit 210 receives an internal hollow and the internal hollow receives the internal rod 250 coupled with the cage 100. The internal rod 250 has a female threaded portion fastened to the pivoting member 120 of the cage 100 at a front end thereof and a rotary knob 252 for a rotating operation at a rear end thereof.

The first moving unit 220 is slidably installed in the support unit 210. The first moving unit 220 is configured to push one side of the first structure reinforcing unit 117 of the cage 100 as the first moving unit 220 moves forward from the support unit 210, that is, moves a direction toward the cage 100.

The second moving unit 230 is slidably installed in the support unit 210 in parallel to the first moving unit 220 and the second moving unit 130 moves backward when the first moving unit 220 moves forward so as to implement an articulated rotary motion of the cage 100, so-called articulating mechanism.

The adjustment unit 240 is installed in the housing so as to be operable by a user and adjusts movement of the first moving unit 220 and the second moving unit 230 according to an operation by the user. In the case of the embodiment, it is exemplified that the adjustment unit 240 has a shape of a rotation handle rotatably installed at the rear end of the support unit 210. Therefore, when the adjustment unit 240 rotates in one direction, the second moving unit 230 may be configured to move backward and a pivoting member 120 may be configured to move forward and when the adjustment unit 240 rotates in an opposite direction thereto, the second moving unit 230 may be configured to move forward and a pivoting member 120 may be configured to move backward. Such a mechanism may be implemented by power transmission by a power transmission unit embedded in the support unit 210.

A fixation unit 260 for fixing and releasing the position of the second moving unit 230 and a handle 270 for allowing the user to perform the procedure by being held by hand may be installed in the support unit 210.

When an operating process of the cage inserting device 200, first, the user rotates a rotary knob 252 of the internal rod 200 to fasten the internal rod 250 and the pivoting member 120 of the cage 100 and operates the fixation unit 260 to fix the position of the second moving unit 230. Then, the cage inserting device enters an insertion position of the vertebral body 10 to insert the cage 100.

In the initial process of insertion of the cage 100, the first moving unit 220 supports the first structure reinforcing unit 117 formed in the slot 115 of the support unit 110 and the second moving unit 230 supports the second structure reinforcing unit 118. Since the position of the second moving unit 230 is fixed by the fixation unit 260, the cage 100 may be stably entered without being arbitrarily rotated by a strong support force when the cage 101 is initially inserted.

When fixation by the fixing unit 260 is released after the initial insertion of the cage 100, the second moving unit 230 is movable. When the adjustment unit 240 rotates in such a state, the first moving unit 220 moves forward to push the first structure reinforcing unit 117 of the support unit 110 and the second moving unit 230 moves backward to provide a rotational space of the cage body 111, and as a result, the articulated rotary motion of the cage 100 is achieved.

When the adjustment unit 140 continuously rotates, the cage 10 rotates to approximately 90 degrees and the second structure reinforcing unit 118 of the cage 100 contacts a lateral surface of the pivoting member 120 to restrict the rotation and in such a state, the cage 100 may be inserted between the vertebrae.

When the insertion of the cage 100 is completed, the user rotates the rotary knob 152 in the direction opposite to the rotational direction at the time of fastening, the internal rod 250 is separated from the pivoting member 120 of the cage 100 and the age inserting device is pulled out from a procedure position.

FIG. 7 sequentially illustrates a rotation process of the cage 100 and FIG. 8 illustrates a position and a rotational state of the cage 100 corresponding thereto. In FIG. 7, a point expressed by P indicates a contact point between the first moving unit 220 and the first structure reinforcing unit 117 as the first moving unit 220 of the cage inserting device moves. As described above, the first moving unit 220 of the cage inserting device moves along the inclined first structure reinforcing unit 117 to transmit force, so that the support unit 110 rotates around the pin member 123. In other words, the inclined first structure reinforcing unit 117 serves to convert a forward motion of the first moving unit 220 of the cage inserting device into a rotary motion of the support unit 110.

The second structure reinforcing unit 118 is supported by the second moving unit 130 of the cage inserting device at the time of the initial insertion of the cage 100, so that the support unit 110 may be stably inserted without being arbitrarily rotated and may serve to restrict the rotation of the cage body 210 when the cage body 210 rotates at approximately 90 degrees.

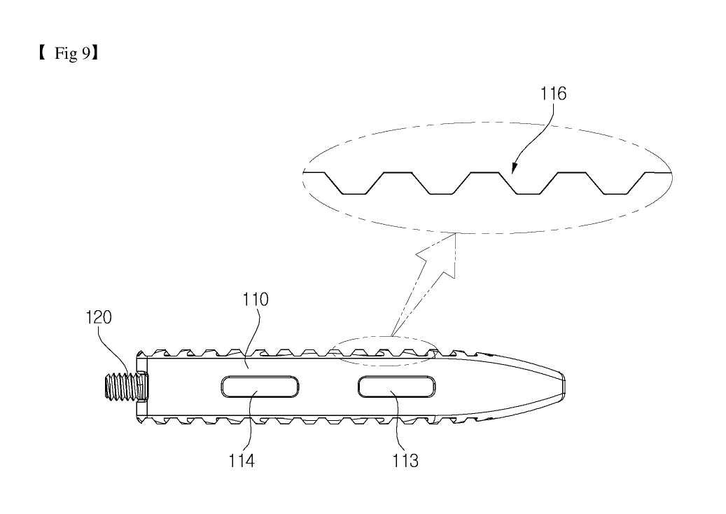

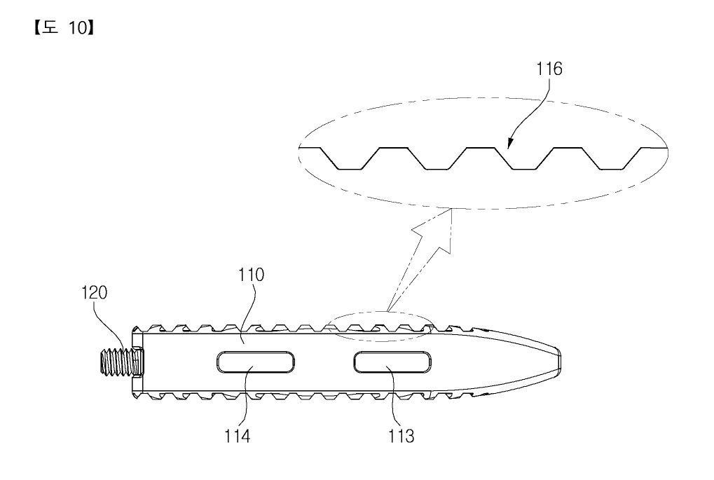

FIG. 9 is a front view of the cage for a disc space between vertebrae, which shows a shape of a concave-convex pattern illustrated in FIG. 1 and FIG. 10 is a front view of the cage for a disc space between vertebrae, which shows a shape of a concave-convex pattern illustrated in FIG. 1.

Referring to FIGS. 9 and 10, a repetitive concave-convex pattern 116 may be formed on the upper and lower surfaces of the support unit 110 and the drawings enlarge and illustrate the structure of the concave-convex pattern 116.

According to the embodiment, the concave-convex pattern 116 is formed in a triangular shape when viewed from the front-rear direction along the insertion direction of the support unit 110 as illustrated in FIG. 9 and is formed in a trapezoidal shape when viewed from a lateral direction vertical to the front-rear direction as illustrated in FIG. 10.

According to such a structure, when a structure is formed in which the concave-convex pattern 116 elongates in the insertion direction at the time of the initial insertion of the cage 100, the concave-convex pattern 116 comes into substantial line contact along the insertion direction. Therefore, the concave-convex pattern 116 serves as a rail, and as a result, the cage 100 may be easily inserted.

In addition, when the cage 100 rotates at approximately 90 degrees to complete the seating between the vertebrae, the concave-convex pattern 116 elongates in a left-right direction between the vertebrae, so that friction force may be increased against movement in the front-rear direction between the vertebrae, that is, a direction toward the back and the abdomen of the human body. Accordingly, sliding of the cage 100, which may be caused by lumbar flexion-extension or the like, is minimized until the bone fusion is completed after the cage 100 is seated, thereby preventing the cage 100 from being separated from the cage 100.

The cage for a disc space between vertebrae described above is not limited to the configurations and methods of the embodiments described above, but all or some of the embodiments may be selectively combined and configured so that various modifications of the embodiments can be made.

What is claimed is:

1. A cage for a disc space between vertebrae, comprising:
a support unit inserted between vertebrae;
a connection portion connected with the support unit, and
a pivoting member including an insertion device connecting unit which extends from an end portion of the connection portion to protrude to the outside of the support unit and connected with an insertion device.

2. The cage for a disc space between vertebrae of claim 2, wherein the connection portion is connected to a cage body by a pin member, and
the pivoting member and the pin member are made of a material having a higher strength than the cage body.

3. The cage for a disc space between vertebrae of claim 1, wherein a slot providing a pivoting space of the pivoting member is formed at a rear end of the support unit, and
a first structure reinforcing unit supported by a first moving unit of the cage inserting device and a second structure reinforcing unit supported by a second moving unit of the cage inserting device are formed in the slot.

4. The cage for a disc space between vertebrae of claim 3, wherein the first and second structure reinforcing units are formed to have predetermined angels, respectively with respect to a coupling direction of the cage inserting device.

5. The cage for a disc space between vertebrae of claim 3, wherein the first structure reinforcing unit is formed to be inclined at an angle of 45 degrees with respect of the coupling direction of the cage inserting device, and
the second structure reinforcing unit is formed to be inclined at an angle of 90 degrees with respect of the coupling direction of the cage inserting device.

6. The cage for a disc space between vertebrae of claim 1, wherein first and second markers having a pin shape are installed in the support unit, and
the pint member is disposed in a triangular shape together with the first and second markers to serve as a positioning marker.

7. The cage for a disc space between vertebrae of claim 1, wherein a concave-convex pattern is repeatedly formed on the surface of the cage body.
